(19) United States
(12) Patent Application Publication
(10) Pub. No.: US 2009/0171272 A1
(43) Pub. Date: Jul. 2, 2009

(54) DEFLECTABLE SHEATH AND CATHETER ASSEMBLY

(76) Inventors: Troy T. Tegg, Elk River, MN (US); Richard E. Stehr, Stillwater, MN (US); William M. Sutton, Minnetonka, MN (US)

Correspondence Address: SJM/AFD - DYKEMA;c/o CPA Global, P.O. Box 52050, Minneapolis, MN, 55402, US

(21) Appl. No.: 11/967,162

(22) Filed: Dec. 29, 2007

(51) Int. Cl. A61M 25/092; A61M 25/01

(52) U.S. Cl. 604/95.04; 604/525

(57) ABSTRACT

The present invention relates to deflectable access sheath assembly. The present invention relates to deflectable sheaths and catheters, including a deflectable sheath access device or introducer, wherein the deflection and movement of the sheath access device is controlled through the relative movement of a plurality of sheaths with respect to one another. Moreover, the present invention relates to catheter assemblies designed for increased flexibility.

### BACKGROUND OF THE INVENTION

a. Field of the Invention

The present invention relates to a deflectable sheath and assembly, including a deflectable sheath access device or introducer, wherein the deflection and movement of the sheath access device is controlled through the relative movement of a plurality of sheaths with respect to one another. Moreover, the present invention relates to catheter assemblies designed for increased flexibility.

b. Background Art

Many medical procedures require the introduction of specialized medical devices into and/or around the human heart. In particular, there are a number of medical procedures that require the introduction of specialized devices including, but not limited to, catheters, dilators, and needles to areas, such as into the atria or ventricles to access the inner surface of the heart, or into the pericardial sac surrounding the heart to access the epicardial or outer surface of the heart. Catheters and access sheaths or introducers have been used for medical procedures for a number of years. It is typically necessary for introducers and catheters to exhibit a degree of flexibility to be able to maneuver through the vasculature of a patient during the performance of cardiac procedures. In additional, various configurations of introducers are necessary for the treatment of different cardiac conditions.

Traditional catheters and access devices, such as introducers, access endocardial areas through a rigid elongated body that includes a curved portion for accessing areas of the heart and related vasculature for ablation. Conventional sheaths, introducers, and catheters are commonly configured with pull wires to control the movement and relative curvature of the devices. The movement of these deflectable devices is primarily controlled by at least one pull wire that is provided about or within the wall of the devices. The pull wires extend along the length of the sheath and are coupled and/or connected to a control mechanism, such as, for example, a knob that can be rotated, which results in the defection of the elongated body of the sheath, introducer or catheter.

For some applications, a steerable sheath (or other access device) comprise an elongated body that may become difficult to control as the elongated is extended and becomes longer. In particular, the pull wires that extend along the length of the sheath are commonly controlled by a mechanism that is provided within a handle at or about the proximal end of the sheath. In various procedures, such as in the treatment of atrial fibrillation or ventricular tachycardia, it is necessary to place this access sheath within the right or left atrium for insertion of a catheter. Accordingly, the movement of the sheath in combination with the catheter must be controlled to a high degree of precision due to the sensitivity of the procedures. One problem often encountered with prior access sheaths and access devices is that once the sheath or device becomes deflected, it may become difficult to apply torque to the sheath or access device without whipping the pull wires provided in or along the wall of the sheath.

In addition to the more rigid deflectable access sheaths and introducers, catheter assemblies provided within the access sheath for access to the target tissue are provided with a tip portion that is traditionally more rigid in order to be manipulated into place. During an intra-cardial procedure, such as those used to treat atrial fibrillation or ventricular tachycardia, there is always a risk for dissection of the tissue with the catheters and the tools being used to perform the procedure. In addition, with the increased use of robotic manipulation systems for the catheters, it can become necessary to provide distal tip sections that are safe to deploy as a user may no longer have direct feedback provided by sensations associated with touch. Currently, there are some catheters that employ a removable inner member to stiffen the catheter for easier insertion through the introducer. Such a stiffer inner member can be removed to soften the distal portion of the catheter. One challenge associated with these types of catheters is that the removable inner member may take up valuable room inside the catheter device and ultimately an extra component must be dealt with by the user and eventually disposed of.

Current access sheath devices and catheter assemblies attempt to maintain control and rigidity through the use of known control mechanisms, i.e., for example, pull wires. Each of these traditional devices may present challenges with being maneuvered and providing catheter tips that are rigid enough to be manipulated into place while at the same time being readily deflectable and soft enough to prevent damage to the sensitive tissue.

### BRIEF SUMMARY OF THE INVENTION

The present invention is directed to deflectable access sheaths or introducers, including a deflectable sheath access device. The present invention includes a deflectable sheath access device or introducer in which the deflection and movement of the sheath access device can be controlled via the relative movement of a plurality of sheaths to one another. In particular, the deflectable sheath access device of the present invention includes a primary sheath, a secondary sheath, and a controller that operably work together to control the positioning and movement of the access device.

Accordingly, embodiments of the present invention provide a deflectable sheath access device. The device includes a primary sheath having a proximal portion and a distal portion having a distal end. The primary sheath further includes a lumen that extends through the length of the primary sheath. The distal portion of the primary sheath further includes a fixed curve. The secondary sheath of the present invention is defined by a longitudinal axis and further includes a proximal end. The secondary sheath (which may also be referred to as a “secondary straight sheath”) is disposed about the proximal portion of the primary sheath. The device of the present invention further includes a controller that includes a control member coupled to the proximal end of the secondary sheath, and controls the relative axial movement of the secondary sheath along the longitudinal axis of the secondary sheath to control the movement of the secondary sheath relative to the primary sheath.

The present invention further provides a deflectable access assembly. The assembly may be used to performing a number of medical procedures. The assembly includes a deflectable sheath access device having a primary sheath, a secondary sheath, and a controller. The primary sheath includes a proximal portion and a distal portion having a distal end. The primary sheath further includes a lumen that extends through the length of the primary sheath. The distal portion of the primary sheath may further include a fixed curve. The secondary sheath of the present invention includes a longitudinal axis and a proximal end. The secondary sheath is disposed about the proximal portion of the primary sheath. The device of the present invention further includes a controller having a control member that is coupled to the proximal end of the secondary sheath and controls the relative axial movement of the secondary sheath along the longitudinal axis of the secondary sheath. This axial movement enables the secondary sheath to move relative to the active movement of the primary sheath. The assembly further includes a catheter comprising a catheter shaft having a distal end portion and a proximal portion, wherein an ablation electrode is coupled to the distal portion of the shaft, for insertion into the passageway defined by the deflectable sheath access device.

The present invention further provides various embodiment of catheter assemblies. In particular, the present invention provides embodiments of catheter assemblies that may be used in connection with the access device of the present invention. Moreover, the catheter assembly, as provided by the present invention, have increased flexibility for use in connection with an access device. The catheter shaft of the present invention includes a distal end portion and a proximal portion. The distal portion may have a durometer less than the proximal portion of the catheter shaft. Furthermore, the present invention may further provide a catheter shaft comprised of a material having a durometer less than traditional catheters, therein providing a flexible or floppy tipped catheter.

Accordingly, deflectable sheath access devices as provided by the present invention, as well as the related assemblies, may be provided to enhance and perform the method of ablating epicardial surface for the treatment of atrial fibrillation and ventricular tachycardia, while readily controlling the movement of the access sheath assembly.

The foregoing and other aspects, features, details, utilities, and advantages of embodiments of the present invention will be apparent from reading the following description and claims, and from reviewing the accompanying drawings.

### BRIEF DESCRIPTION OF THE DRAWINGS

FIG. 1 is an isometric view of deflectable sheath access assembly according to an embodiment of the present invention;

FIG. 2 is an isometric view of deflectable sheath access device according to an embodiment of the present invention;

FIG. 3 is a an isometric view of portion of the deflectable sheath access device according to an embodiment of the present invention;

FIG. 4 is a an isometric view of portion of the deflectable sheath access assembly according to an embodiment of the present invention;

FIG. 5 is an isometric view of deflectable sheath access assembly according to an embodiment of the present invention illustrating a relative starting position in phantom; and

FIGS. 6-9 are cross-sectional partial views of catheter assemblies in accordance with various embodiments of the present invention.

### DETAILED DESCRIPTION OF THE INVENTION

In general, the present invention relates to a deflectable sheath access device or introducer for use in epicardial procedures, such as those used for the treatment of atrial fibrillation and ventricular tachycardia. Furthermore, the present invention relates to a catheter assembly for use with the deflectable sheath access device that includes a relatively flexible tip portion. The deflectable access device provides a primary sheath and a secondary sheath for controlling movement and relative positioning of the deflectable sheath access device, for example, to epicardial surfaces for performing ablation. For purposes of this description, similar features among the various embodiments described herein will be referred to by the same reference number. As will be appreciated, however, the structure of the various aspects may differ with respect to alternate embodiments.

The deflectable access device, or introducer, of the present invention is configured such that pull wires are not necessary to control the relative movement and positioning of the access device. Even with the absence of pull wires, the present invention enables a degree of curvature of the access device to be maintained and modified through the relative movement of access sheaths to one another.

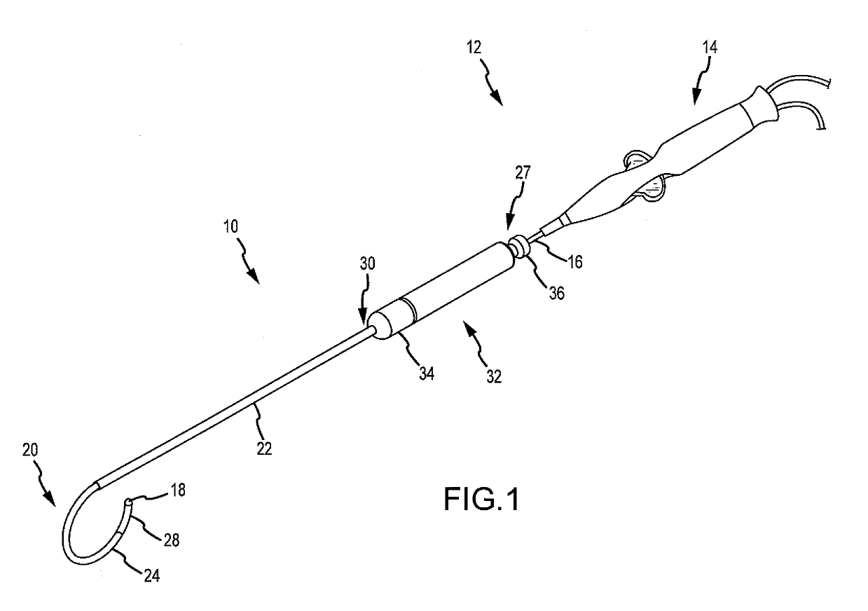

As generally shown in the embodiment illustrated in FIG. 1, the sheath access device 10 may comprise part of a deflectable sheath access assembly 12. The deflectable sheath access assembly 12 may be configured to facilitate access to the epicardial surface of the heart through the pericardial sac or any other area of the heart that are traditionally accessed during the performance of various ablation or mapping procedures. Alternately, assembly 12 may be used, for example, for a number of different procedures. In an embodiment, the deflectable sheath access device assembly 12 includes sheath access device 10 in connection with or coupled to a catheter assembly 14. Catheter assembly 14 provides a catheter shaft 16 having an electrode 18 connected to the distal end portion of catheter shaft 16. Catheter shaft further includes proximal portion 19. A catheter assembly 14, as provided in accordance with embodiments of the present invention, is further described below. Device 10 can be integrated with catheter assembly 14 to provide an operable access assembly 12, which is overall absent of pull wires and wherein the movement of the device is principally controlled through the relative positioning of various components of device 10 and assemblies 12, 14, for example, as described below.

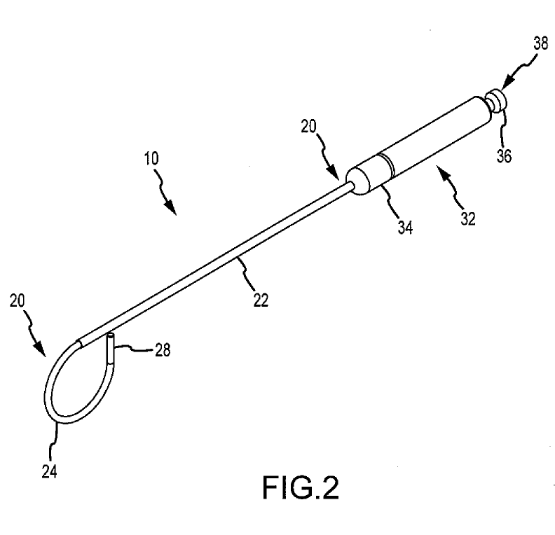

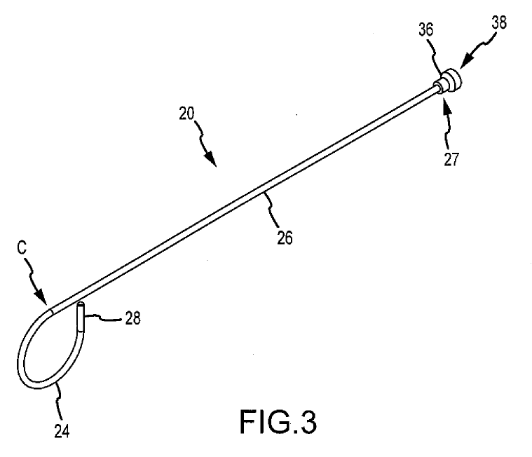

In accordance with an embodiment, as generally illustrated in FIGS. 1 and 2, device 10, also referred to as an introducer, comprises a primary sheath 20 and a secondary sheath 22. Primary sheath 20 includes a distal portion 24 and a proximal portion 26 (e.g., as shown in FIG. 3). Primary sheath 20 further includes proximal end 27 which is positioned proximal to proximal portion 26, Primary sheath 20 further includes a distal tip portion 28. Distal portion 24 of primary sheath 20 further includes a fixed curve. Distal portion 24 is positioned distally with respect to proximal portion 26 and distal tip portion 28 is positioned distally with respect to distal portion 24. Accordingly, distal portion 24 and proximal portion 26 intersect or interconnect with one another to form the unitary primary sheath 20. Primary sheath 20 further includes an inner lumen (not shown) that allows for the insertion and positioning of various medical tools and devices, including but not limited to, catheters for use in procedures, such as for example, ablation or mapping, dilators, electrode tips, or any other type of devices or tools that may need to be inserted through an access device. In general, access device 10 facilitates the introduction and position of devices to establish operable contact and/or communication with targeted tissue areas.

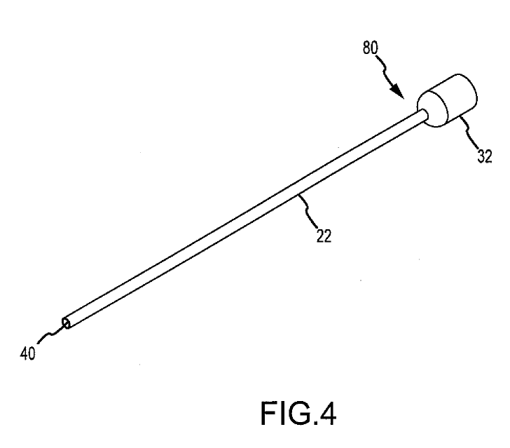

Access device 10 further includes a secondary sheath 22 that is defined by a longitudinal axis. Secondary sheath 22 further includes a proximal end 30. The secondary sheath is substantially straight (and, hence, is also referred to as a “secondary straight sheath” or a “straight sheath”) and the secondary sheath maintains its relative rigidity through the operation and movement of access device 10, as described below. The term “straight” is meant to include substantially or relatively straight in relation to any curved portion or part of primary sheath 20. Secondary sheath 22 may be disposed about proximal portion 26 of primary sheath 20. Accordingly, as illustrated in FIG. 4, secondary sheath 22 further includes a lumen 40 that is configured to receive primary sheath 20, e.g., as shown in FIGS. 1 and 2. Proximal end 30 of secondary sheath 22 is further connected to or coupled with controller 32. Controller (such as, for example, a mechanical device or handle) 32 is connected to or coupled with proximal end 30 of secondary sheath 22. Controller 32 further includes control member 34. Control member 34 is provided on distal end 36 of controller 32, such that control member 34 is connected to or coupled with proximal end 30 of secondary sheath 22. In general, controller 32 controls the relative axial movement of secondary sheath 22 along the longitudinal axis of secondary sheath 22, therein moving secondary sheath 22 relative to primary sheath 20.

Device 10 may further include locking mechanism 36 to secure the relative position through the use of friction of primary sheath 20 and secondary sheath 22. Locking mechanism 36 may be provided at the proximal end 40 of device 10, or, as illustrated in connection with FIG. 3, at proximal end 27 of primary sheath 20. Locking mechanism 36 may further be provided with an aperture 38 for allowing the introduction of medical device components into the inner lumen of the primary sheath 20. FIGS. 2 through 9, illustrate alternate embodiments of device 10 and catheter assembly 14 and depict some exemplary applications of a sheath access device 10 in connection with various catheter assemblies 14.

As can be seen in FIG. 3, primary sheath 20 includes distal portion 24 and proximal portion 26. Primary sheath 20 may further include a distal tip portion 28 that is provided distally with respect to distal portion 24. Primary sheath 20 further includes a proximal end 27 that is provided proximally with respect to proximal portion 26. Primary sheath 20 may be generally comprised of a biocompatible polymer material that exhibits various degrees of flexibility and rigidity, depending on the design and performance requirements. In an embodiment, primary sheath 20 is comprised of a thermoplastic material, such as, without limitation, PEBAX®. Throughout the length of primary sheath 20, various materials such as thermoplastics (e.g. PEBAX®) that exhibit different degrees of hardness (or durometer) may be used. In addition, the material may be braided or provided in alternate configurations to achieve a desired degree or measure of rigidity and/or flexibility. The device may also be lined with Teflon in order to reduce friction during insertion of various instruments. In an embodiment, proximal portion 26 is made of a different material than the material comprising distal portion 24, such that the hardness or durometer of proximal section 26 is greater than the hardness or durometer of distal portion 24. According to an embodiment of the present invention, proximal portion 26 may include a hardness/rigidity, for example, within the range of 60-75 durometer. For some embodiments the range may be 63-72 durometer, and may, for example, be approximately a 72 durometer. For some embodiments, distal portion 24 may include a hardness/rigidity within the range of 22-50 durometer, and may, for example, be approximately a 40 durometer. In another embodiment, distal tip portion 28 may be made of material, such that the hardness/rigidity of distal tip portion 28 is even less than distal portion 24. This softness or flexibility can be provided to prevent damage to the pericardial sac and epicardial surface of the heart. Accordingly, distal tip portion 28 may have a hardness/rigidity within the range of 20-50 durometer, and for some embodiments may be approximately 40 durometer. Moreover, distal tip portion 28 may be conferred to provide a smooth transition from primary sheath 20 to any medical device disposed within the inner lumen of sheath 20. In alternate embodiments, various sections of sheath 20 may be radiopaque or include various fluorescent markers such that the access sheath is visible through fluoroscopy.

Variations in size and shape of primary sheath 20 may be used and are intended to encompass all applicable uses. It is recognized that based on the application and/or use of device 10, the length of primary sheath 20 may vary slightly. Primary sheath 20, in accordance with the present invention, is generally configured to be shorter than the length of traditional catheter assemblies, so that the distal end of the catheter including the electrode may be disposed out of distal tip portion 28, as illustrated in FIG. 1. Accordingly, the length of primary sheath 20 is approximately 10 cm less than a traditional catheter. The length ranges for primary sheath 20 may include from 30-60 inches. The length of curved distal tip portion 28 may range from approximately 1.5 inches to approximately 6 inches in length. Slight variations may be made to the length depending on the design or manufacturing of the system.

As generally illustrated in FIG. 3, distal portion 24 of primary sheath 20 may comprise a fixed curve. More particularly, distal portion 24 can be provided with a fixed curved configuration that may range from approximately 0 degrees to approximately 270 degrees from the relative point of curvature (c) provided on the longitudinal axis of device 10. In an embodiment, the fixed curve may be 270 degrees from the relative point of curvature (c). Depending on the functional and overall design of device 10, the degree of curvature of the fixed curve of primary sheath 20 may vary and/or be modified. Primary sheath 20 may further include a locking mechanism 36 disposed on the proximal end 27 of primary sheath 20.

As previously described and shown in FIG. 4, secondary sheath 22 may be substantially straight and is relatively proportioned to reflect the length of proximal portion 26 of primary sheath 20. Secondary sheath 22 further may include lumen 40 that may slidably receive primary sheath 20. Lumen 40 is relatively proportioned to have a diameter slightly greater than the diameter of primary sheath 20 such that primary sheath 20 may be disposed within lumen 40. Moreover, sufficient clearance is provided between the interface of the surfaces of primary sheath 20 and secondary sheath 22 to allow secondary sheath 22 to be slidably moved along primary sheath 22. Similar to proximal portion 26 of primary sheath 20, secondary sheath 22 may be generally comprised of a biocompatible polymer material that exhibits various degrees of flexibility and rigidity. For example, secondary sheath 22 may be comprised of a thermoplastic material, such as, without limitation, PEBAX®. In addition, the material may be braided or provided in alternate configurations to achieve a desired degree or measure of rigidity and/or flexibility. The device may also be lined with Teflon in order to reduce friction during insertion of primary sheath 20 and the slidable axial movement of primary sheath 20. According to an embodiment of the present invention, secondary sheath 22 may include a hardness/rigidity within the range of 60-75 durometer. For some embodiments the range may be 63-72 durometer, and may, for example, be approximately a 72 durometer. An alternate embodiment of secondary sheath 22 may be provided wherein secondary sheath 22 is disposed within primary sheath 20 instead of being provided on the external surface surrounding primary sheath 20.

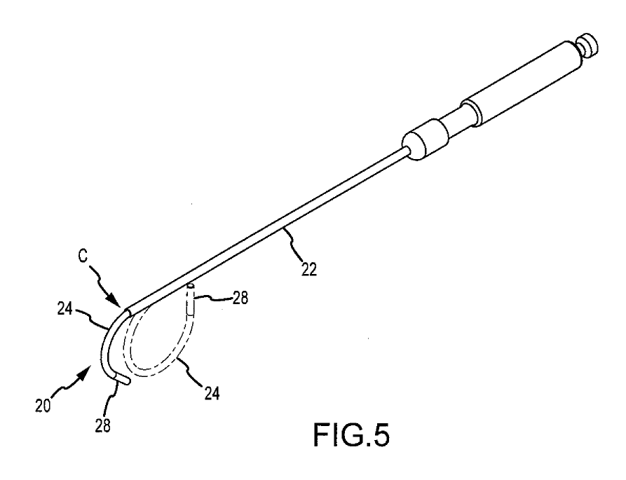

As further shown in FIG. 4, control member 34 of controller 32 is positioned at proximal end 30 of secondary sheath 22. Control member 34 may be moved axially along the longitudinal axis of secondary sheath 22. In particular, as illustrated in FIG. 5, control member 34 may move axially along the longitudinal axis of secondary sheath 22 towards distal tip portion 28 of primary sheath 20. As illustrated in FIG. 5, the original position of the fixed curve of distal portion 24 of primary sheath 20 is shown in phantom. Upon slidably moving control member 34 (i.e., for example, a slid knob), a slidable portion 42 connected to control member 34 can be moved axially within the main body 44 of controller 32 (i.e., for example, a handle). Accordingly, as the control member 34 is slidably moved axially towards distal tip portion 28 of primary sheath 20, slidable portion 42 extends from main body 44 of controller 32. Simultaneously, as control member 32 is moved axially towards distal tip portion 28 of primary sheath 20, the fixed curve of distal member 24 is gradually deflected from the original fixed curve position (i.e., for example, 270 degree) to a lesser degree fixed curve that may, for example, range from 0 degrees (relatively straight distal portion 24 of primary sheath) to less than 270 degrees fixed curve of distal portion 24. As the secondary sheath 22 is moved axially over primary sheath 20, the fixed curve of distal portion 24 may be modified, for instance as generally shown in FIG. 5. Depending on the desired relative change in the degree of the fixed curve provided by distal portion 24, secondary sheath 22 may be moved varying amounts along primary sheath 20. Accordingly, it is the relative movement of straight secondary sheath 22 that controls and manipulates the movement of the fixed curve of distal portion 24 of primary sheath 20. Depending on the degree of curve desired, secondary sheath 22 may be moved varying amounts in relation to and along the longitudinal axis of primary sheath 20.

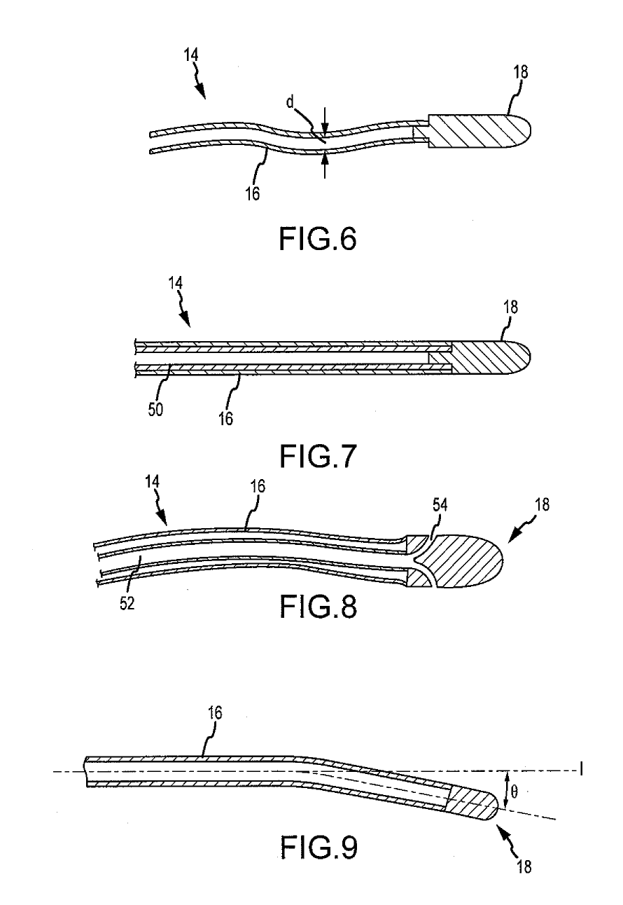

In addition to various embodiments of access device 10 as previously described, the present invention further provide access assembly 12 as shown in FIG. 1. Access assembly 12 further includes catheter assemblies 14 having various embodiments for use within access device 10 not containing pull wires. Catheter assembly 16, as shown in FIG. 1, may be configured to include a handle (as shown in FIG. 1), or may be configured without a handle. FIGS. 6 through 9 illustrate additional embodiments of catheter assemblies 14, including catheter shafts 16 that may be used in connection with device 10 of the present invention.

In accordance with embodiments of the present invention and in connection with the use of the access device 10, it may be desirable to have a catheter shaft 16 and in particular a distal end portion 46 of catheter shaft 16 that has increased flexibility (i.e. floppy) compared to traditional catheter assemblies. In particular, it may be desirable to use softer, more desirable material while at the same time meeting industry standards regarding the rigidity and tensile strength of catheter shafts. Various modifications may be made to provide flexible or floppy tip catheters that perform in accordance with softer materials, while at the same time meeting the tensile strength standards of the industry, as well as any other applicable standards.

Catheter assembly 14, in accordance with an embodiment of the present invention, may provide a catheter shaft 16 that is comprised of a material that has a soft durometer, i.e., for example 25-40 durometer, therein exhibiting increased flexibility in the catheter shaft. Overall, the distal end portion 46 of catheter shaft 16 may have a stiffness range of approximately 0.005 lbs to 0.100 lbs when measured using a 1 inch long section supported at one end and the relative displacement of distal tip section 46 to the longitudinal axis of catheter shaft 16 may be approximately 0.500 inches.

Moreover, as generally illustrated in FIG. 6, the diameter (d) of catheter shaft 16, such as, for example, distal end portion 46, may be relatively smaller in size than traditional catheter shafts. A reduced diameter or reduced cross-section of a catheter shaft increases the flexibility and movement of catheter assembly 14, therein allowing the assembly 14 to be more readily moved and controlled through the relative position of access device 10. Catheter shaft 16 (e.g., as shown in FIG. 6) may include the hardness, or durometer, of a traditional catheter shaft, such as, for example, 40-80 durometer, or may be provided having a reduced hardness, such as 25-50 durometer. The diameter (d) of catheter shaft 16 may range from 4-6 french in size, compared to a traditional 7 french electrode tip 18.

In an embodiment, as generally shown in FIG. 7, catheter assembly 14 may further include a lower durometer inner shaft 50 disposed within catheter shaft 16. The lower durometer inner shaft 50 may have a reduced hardness, such as 25-50 durometer. The incorporation of inner shaft 50 provides increase flexibility to the catheter assembly 12, even though catheter shaft 16 may be comprised of a more durable bio-compatible material, such as braided PEBAX®, having an increased durometer than inner shaft 50. Catheter shaft 16 may be comprised of material having a durometer of approximately 72 durometer, such a more durable braided shaft. Overall, catheter assembly 14 exhibits increased flexibility based on the incorporation of inner shaft 50.

As further illustrated in FIG. 8, catheter assembly 14 may include a catheter shaft 16 having increased flexibility and a relatively softer durometer, i.e., for example, 25-50 durometer, than traditional catheter shafts. In accordance with this embodiment, catheter shaft 16 may further include fluid lumen 52 disposed within catheter shaft 16 and running the length of catheter shaft to electrode tip 18. In order to increase flexibility and strength, fluid lumen 52 may be braided. Catheter assembly 14 further provides irrigation passageways 54. Fluid lumen 52 may be provided having a softer durometer, such as 25-50 durometer. The softer fluid lumen 52 in combination with a softer catheter shaft 16 allow for increased flexibility of catheter assembly 14, in particular, the irrigated catheter assembly 14. An alternate embodiment of catheter assembly 14, may further provide a closed-end fluid lumen that may be pressurized through the use of fluid in order to add stiffness to catheter shaft 16 upon insertion of catheter assembly 14 within access device 10 or any other time that additional stiffness is necessary.

Another embodiment of catheter assembly 14, as generally shown in FIG. 9, illustrates an example of a catheter assembly 14 having a distal end 46 that is deflected from the longitudinal axis (l) of catheter shaft 16 by an angle (Θ) of approximately 10-15 degrees. In order to maintain the desired angle of distal end 46 relative to the longitudinal axis of catheter shaft 16, traditional catheter shafts having standard rigidity and flexibility may be used in accordance with the present embodiment. The catheter shaft 16 of assembly 14 provides an offset angle may predispose the catheter to buckle or respond to external pressure exerted by the catheter assembly 14, instead of perforating or puncturing the tissue surface.

Although not illustrated, each of the provided embodiment of catheter shaft 16 may further include a reinforced section that may, for example, be provided within distal section 46 of catheter 16. Such a reinforced section (or segment) may employ or include a glass fiber braid that has extremely high tensile strength and increase flexibility. The glass fiber braid may be selected form various fibers, including Vectran®. Moreover, catheter assemblies having tip electrodes may use an auxiliary wire fastened to the electrode tip for safety. In order to maintain tensile strength in the flexible (or floppy) catheters while at the same time keeping the wire flexible, a braided safety wire can be used. A braided safety wire may be braided from 0.0010-0.0015″ metal wire to make a high flexible high tensile strength braided assembly.

Embodiments of flexible catheters, including those with catheter shafts as generally illustrated and discussed above, may be readily incorporated with an access device 10 for performing ablative procedures. Other types of energy sources may also be used in connection with access sheath device 10 of the present invention, such as ultrasound (e.g. HIFU), laser, or other energy used for performing ablative procedures. Additional electrode tips may be used and configured, such as a closed loop cooled tip, for incorporation with the shorted catheter assembly for insertion within access sheath device 10.

An assembly or kit for use in treating various conditions, may include deflectable access sheath device 10 and catheter assembly 14, as such described in accordance with the multiple embodiments of the present invention.

Although a number of embodiments of this invention have been described above with a certain degree of particularity, those skilled in the art could make numerous alterations to the disclosed embodiments without departing from the spirit or scope of this invention. All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A deflectable sheath access device, the device comprising the following:
a primary sheath having a proximal portion, a distal portion having a distal end, and a lumen extending through the primary sheath, the distal portion of the primary sheath includes a fixed curve,
a secondary straight sheath including a longitudinal axis and having a proximal end, the secondary straight sheath is disposed about the proximal portion of the primary sheath; and
a controller including a control member that is connected or coupled to the proximal end of the secondary straight sheath and controls the relative axial movement of the secondary straight sheath along the longitudinal axis of the secondary straight sheath to move the secondary straight sheath relative to the primary sheath.

2. The device of claim 1, wherein the fixed curve of the primary sheath is approximately a 270 degree curve.

3. The device of claim 1, wherein the secondary straight sheath is shorter than the primary sheath.

4. The device of claim 1, wherein the secondary sheath is configured to slidably move towards the distal portion of the primary sheath to deflect the fixed curve of the distal portion of the primary sheath towards the longitudinal axis of the secondary sheath.

5. The device of claim 4, wherein the secondary sheath is configured to slidably move away from the distal portion of the primary sheath therein deflecting the fixed curve of the distal portion of the primary sheath away from the longitudinal axis of the secondary sheath.

6. The device of claim 1, wherein the controller further includes a slidable portion connected to the control member, and the slidable portion is received within an outer body member.

7. The device of claim 1, wherein the fixed curve of the primary sheath may range from 270 degrees to 0 degrees of curvature.

8. The device of claim 1, wherein the primary sheath is comprised of a braided thermoplastic.

9. The device of claim 1, wherein the secondary straight sheath is comprised of a braided thermoplastic.

10. The device of claim 1, wherein the secondary straight sheath is more rigid than the distal portion of the primary sheath.

11. The device of claim 1, where in the primary sheath further includes a locking mechanisms for securing the secondary sheath in a relative position to the primary sheath.

12. The device of claim 11, wherein the locking mechanism further includes an aperture that is coupled to the lumen provided within primary sheath therein providing a passageway.

13. A deflectable access assembly for use in performing medical procedures, the assembly comprising the following:
deflectable sheath access device including a primary sheath, a secondary straight sheath, and a controller,
the primary sheath having a proximal portion, a distal portion having a distal end, and a lumen extending through the primary sheath, the distal portion of the primary sheath includes a fixed curve;
the secondary straight sheath including a longitudinal axis and having a proximal end, and disposed about the proximal portion of the primary sheath; and
the controller having an inner cavity and a control member that is connected or coupled to the proximal end of the secondary sheath and controls the relative axial movement of the secondary straight sheath along the longitudinal axis of the secondary sheath therein moving the secondary sheath relative to the primary sheath; and
a catheter comprising a catheter shaft having a distal end portion and a proximal portion, wherein an ablation electrode is coupled to the distal portion of the shaft, for insertion into the passageway defined by the deflectable sheath access device.

14. The assembly of claim 13, wherein the distal end portion of the catheter shaft has a durometer less than the proximal portion of the catheter shaft.

15. The assembly of claim 14, wherein the proximal portion of the catheter shaft includes a glass fiber braided portion.

16. The assembly of claim 13, wherein the catheter further includes a fluid lumen disposed within the catheter shaft.

17. The assembly of claim 16, wherein the fluid lumen is braided.

18. The assembly of claim 14, wherein the catheter includes an inner shaft having a hardness less than the catheter shaft.

19. The assembly of claim 14, wherein the distal end portion of the catheter is deflected from a longitudinal axis of the catheter shaft.

20. A catheter assembly having increased flexibility for use in connection with an access device, the assembly comprising:
a catheter shaft having a distal end portion; and
a proximal portion,
wherein the distal portion has a durometer less than the proximal portion of the catheter shaft.

21. The assembly of claim 20, wherein the proximal portion of the catheter shaft includes a glass fiber braided portion.

22. The assembly of claim 20, wherein the catheter further includes a fluid lumen disposed within the catheter shaft.

23. The assembly of claim 22, wherein a wall defining the fluid lumen is braided.

24. The assembly of claim 20, wherein the catheter includes an inner shaft having a hardness less than the catheter shaft.

25. The assembly of claim 20, wherein the distal end portion of the catheter is deflected from a longitudinal axis of the catheter shaft.
